SEQUENCE LISTING (24 sequences)

SEQ ID NO: 1 - protein, 1043 aa, synthetic construct
  source 1..1043: note = sequence of secretion tag for trypsin
GGSQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRA

SEQ ID NO: 2 - protein, 15 aa, synthetic construct
  source 1..15: note = sequence of secretion tag for human interleukin 2
MNPLLILTFVAAALA

SEQ ID NO: 3 - protein, 20 aa, synthetic construct
MYRMQLLSCIALSLALVTNS

SEQ ID NO: 4 - protein, 18 aa, synthetic construct
  source 1..18: note = sequence of secretion tag for serum albumin
MKWVTFISLLFLFSSAYS

SEQ ID NO: 5 - protein, 19 aa, synthetic construct
  source 1..19: note = sequence of secretion tag for disulfide-bond formation protein A
MKKIWLALAGLVLAFSASA

SEQ ID NO: 6 - protein, 22 aa, synthetic construct
  source 1..22: note = sequence of secretion tag for pectate lyase B
MKYLLPTAAAGLLLLAAQPAMA

SEQ ID NO: 7 - protein, 21 aa, synthetic construct
  source 1..21: note = sequence of secretion tag for outer membrane protein A
MKKTAIAIAVALAGFATVAQA

SEQ ID NO: 8 - protein, 26 aa, synthetic construct
  source 1..26: note = sequence of secretion tag for maltose-binding protein
MKIKTGARILALSALTTMMFSASALA

SEQ ID NO: 9 - protein, 20 aa, synthetic construct
  source 1..20: note = sequence of secretion tag for murein lipoprotein
MKATKLVLGAVILGSTLLAG

SEQ ID NO: 10 - protein, 46 aa, synthetic construct
  source 1..46: note = sequence of secretion tag for trimethylamine N-oxide reductase
MNNNDLFQASRRRFLAQLGGLTVAGMLGPSLLTPRRATAAQAATDA

SEQ ID NO: 11 - protein, 25 aa, synthetic construct
  source 1..25: note = sequence of secretion tag for mannosidase
MRLLTALFAYFIVALILAFSVSAKS

SEQ ID NO: 12 - protein, 24 aa, synthetic construct
  source 1..24: note = sequence of secretion tag for human insulin
MALWMRLLPLLALLALWGPDPAAA

SEQ ID NO: 13 - protein, 367 aa, synthetic construct
  source 1..367: note = maltose-binding protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 14 - protein, 212 aa, synthetic construct
  source 1..212: note = sequence of cysteine protease
MALADGKILHNQNVNSWGPITVTPTTDGGETRFDGQIIVQMENDPVVAKAAANLAGKHAESSVVVQLDSDGNYRVVYGDPSKLDGKLRWQLVGHGRDHSETNNTRLSGYSADELAVKLAKFQQSFNQAENINNKPDHISIVGCSLVSDDKQKGFGHQFINAMDANGLRVDVSVRSSELAVDEAGRKHTKDANGDWVQKAENNKVSLSWDAQG

SEQ ID NO: 15 - protein, 218 aa, synthetic construct
  source 1..218: note = sequenceof glutathione S-transferase
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 16 - protein, 201 aa, synthetic construct
  source 1..201: note = sequenceof apolipoprotein A1
MLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 17 - protein, 109 aa, synthetic construct
  source 1..109: note = sequence of thioredoxin
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 18 - protein, 232 aa, synthetic construct
  source 1..232: note = green fluorescent protein
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHG

SEQ ID NO: 19 - protein, 7 aa, synthetic construct
  source 1..7: note = tobacco etch virus cleavage sequence
ENLYFQG

SEQ ID NO: 20 - protein, 6 aa, synthetic construct
  source 1..6: note = thrombin cleavage sequence
LVPRGS

SEQ ID NO: 21 - protein, 5 aa, synthetic construct
  source 1..5: note = enterokinase cleavage sequence
DDDDK

SEQ ID NO: 22 - protein, 4 aa, synthetic construct
  source 1..4: note = factor Xa cleavage sequence
IEGR

SEQ ID NO: 23 - protein, 8 aa, synthetic construct
  source 1..8: note = rhinovirus 3C cleavage sequence
LEVLFQGP

SEQ ID NO: 24 - DNA, 3948 bp, synthetic construct
  source 1..3948: mol_type = other DNA; note = sequence of encoding gene is inserted between two cleavage sites NheI and EcoRI of the base vector
atgagactgctgaccgccctgttcgcctacttcatcgtggccctgatcctggccttcagcgtgtccgccaagagcatgcaccaccaccatcaccatcatcacatgagcaagggcgaggaactgttcaccggcgtggtgcccatcctggtggaactggacggcgacgtgaacggccacaagttctctgtgcggggcgagggcgagggggacgccacaaatggcaagctgaccctgaagttcatctgcaccaccggaaagctgcccgtgccctggcctaccctggtcacaaccctgacctacggcgtgcagtgcttcagcagataccccgaccacatgaagcggcacgatttcttcaagagcgccatgcccgagggctacgtgcaagaacggaccatcagcttcaaggacgacggcacctacaagaccagagccgaagtgaagttcgagggcgacaccctggtcaaccggatcgagctgaagggcatcgacttcaaagaggacggcaacatcctgggccacaagctggaatacaacttcaacagccacaacgtgtacatcaccgccgacaagcagaagaacggcatcaaggccaacttcaagatccggcacaacgtggaagatggcagcgtgcagctggccgaccactaccagcagaacacccccatcggcgacggccccgtgctgctgcccgacaatcactacctgagcacccagagcgtgctgagcaaggaccccaacgagaagcgggaccacatggtgctgctggaatttgtgaccgccgctggcatcacccacggcgagaacctgtacttccaagggggatcccagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggcttaa

(19) United States
(12) Patent Application Publication
(10) Pub. No.: US 2025/0051422 A1
(43) Pub. Date: Feb. 13, 2025

(54) RECOMBINANT COLLAGEN, EXPRESSION METHOD AND USE THEREOF

(71) Applicant: Shandong D-nutrimec Biomedical Co., Ltd., Heze City (CN)

(72) Inventors: Zhidong GUO, Heze City (CN); Jiajia SHAO, Heze City (CN); Hongwei MA, Heze City (CN); Huawei AI, Heze City (CN)

(21) Appl. No.: 18/507,236

(22) Filed: Nov. 13, 2023

(30) Foreign Application Priority Data
Aug. 9, 2023 (CN) 2023110002962

(51) Int. Cl. C07K 14/78; A61K 9/00; C12N 9/24; C12N 15/85; C12P 21/00

(52) U.S. Cl. CPC C07K 14/78; A61K 9/0014; C12N 9/2402; C12N 15/85; C12P 21/005; C12Y 302/01024; A61K 38/00; C12N 2800/107

(57) ABSTRACT

Provided are a recombinant collagen, an expression method and use thereof. In the present disclosure, the recombinant collagen is expressed by a human embryonic kidney cell Expi293F at a high protein expression level, thereby realizing the expression of a large-molecular-weight recombinant collagen. Activity studies have showed that the expressed recombinant collagen has desirable activity in promoting cell migration, while the recombinant collagen III+I with a fusion tag shows high stability at 4° C. In addition, cell activity studies have shown that the recombinant type III collagen expressed by human cells has better activity in promoting the cell migration than that of a commercially available type III collagen. These results indicate that the human cell expression system is conducive to the expression of highly active and large-molecular-weight recombinant collagen.

### TECHNICAL FIELD

The present disclosure belongs to the technical field of biochemical industry, and in particular relates to a recombinant collagen, an expression method and use thereof.

### BACKGROUND

Collagen is a biological macromolecular protein ubiquitous in animals, accounting for about 25% to 30% of the total protein in mammals. The collagen is found in different tissues, including musculoskeletal tissues as bones, tendons, and ligaments, and non-musculoskeletal tissues such as cornea, blood vessels, and skin.

The collagen family includes 28 members containing at least one triple-helical domain, and these members interact with cells through several receptor families to regulate cell proliferation, migration, and differentiation. All members have a triple-helical structure, and peptide chains that make up the triple-helical structure are called a chains. The three peptide chains can be identical to form a homotrimer or different to form a heterotrimer.

Type I collagen has a typical feature of the collagen family: a trimeric structure with a repeating sequence of Gly-X-Y, where X and Y are generally proline and 4-hydroxyproline, respectively. Type I collagen is composed of two identical al chains and one α2 chain, and the polypeptide chains are alternately wound in a right-handed helix conformation to form a superhelical structure. A stable triple-helical structure is formed due to hydrogen bonding and electrostatic interactions between repeat sequences and chains.

Collagen is widely used in the fields of medicine, food, and beauty. Due to multiple advantages such as biocompatibility and biodegradability, collagen can be used as medical biomaterials and used in cosmetics. For example, collagen can be used to prevent bacterial infection and relieve pain in skin scalds and burns, can be made into injection solutions to achieve cosmetic effects, and can also be used as drug carriers, bones, scaffold materials, and heart valves. At present, there is still much research on the collagen, such as predicting fibrosis by measuring a collagen content in organs. Moreover, the collagen can also be combined with other drugs as potential diagnostic biomarkers and therapeutic targets to mediate cell migration, and is expected to expand its frontier applications.

There are various methods of extracting collagen. Currently, animal-derived collagen is mainly extracted by acid method, alkali method, and enzymatic method, which can obtain the complete triple-helical structure of collagen to maintain its biological characteristics, and has low cost and high economic value. However, animal-derived collagen still has many disadvantages such as immunogenicity, poor uniformity, and hidden dangers of infectious diseases. In contrast, recombinant collagen prepared by genetic engineering technology reduces the risk of rejection, immunogenicity, and virus infection compared with animal-derived collagen, and is currently the most potential method to solve the problem of collagen source. Therefore, the recombinant collagen is a hot spot in collagen production research.

Till now, collagen is mostly prepared and expressed by Escherichia coli, Saccharomyces cerevisiae, and Pichia pastoris. However, Escherichia coli is difficult to express soluble collagen, easily forms inclusion bodies during expression, and shows high difficulty in product purification. Full-length collagen has a larger molecular weight and various post-translational modifications. The post-translational modification of collagen in a yeast system is quite different from that in the human body, which is difficult to replace full-length human collagen.

### SUMMARY

An objective of the present disclosure is to provide a recombinant collagen, and an expression method and use thereof. The expression method significantly increases expression level of the recombinant collagen, and the recombinant collagen has a desirable activity in promoting cell migration and high stability.

The present disclosure provides a recombinant collagen, including all or a part of amino acid sequences of type III human collagen and all or a part of amino acid sequences of type I human collagen.

Preferably, the part of amino acid sequences of the type III human collagen includes amino acid sequences positions 847 to 849 or at positions 154 to 1,221 of type III human collagen; and

part of amino acid sequences of the type I human collagen includes amino acid sequences at positions 80 to 1,119 of an α2 amino acid sequence of the type I human collagen.

Preferably, the recombinant collagen further includes a secretion tag, a histidine tag, a fusion tag, and a protease cleavage site sequence.

Preferably, the secretion tag is selected from the group consisting of trypsin, human interleukin 2, serum albumin, disulfide-bond formation protein A, pectate lyase B, outer membrane protein A, maltose-binding protein (whose coding gene is MalE), murein lipoprotein, trimethylamine N-oxide reductase, mannosidase, human insulin, and a hybrid secretion tag;

the fusion tag is selected from the group consisting of maltose-binding protein (MBP), cysteine protease, glutathione S-transferase, apolipoprotein A1, thioredoxin, green fluorescent protein, and a hybrid fusion tag; and

the protease cleavage site sequence is selected from the group consisting of a tobacco etch virus cleavage sequence, a thrombin cleavage sequence, an enterokinase cleavage sequence, a factor Xa cleavage sequence, a rhinovirus 3C cleavage sequence, and a hybridase cleavage site sequence.

Preferably, the recombinant collagen includes an amino acid sequence shown in SEQ ID NO: 1.

The present disclosure further provides a recombinant expression vector, including an encoding gene of the recombinant collagen.

Preferably, the encoding gene includes a nucleotide sequence shown in SEQ ID NO: 24.

The present disclosure further provides an expression method of the recombinant collagen, including: transforming the recombinant expression vector into a human embryonic kidney cell Expi293F, culturing and expressing human embryonic kidney cell Expi293F.

The present disclosure further provides use of the recombinant collagen or a recombinant collagen prepared by the expression method in preparation of a product for promoting a cell migration activity.

The present disclosure further provides use of the recombinant collagen or a recombinant collagen prepared by the expression method in preparation of a skin repair product.

Beneficial effects: the present disclosure provides a recombinant collagen, which is obtained by splicing amino acid sequences of type III human collagen and an α2-chain of a type I human collagen. In the present disclosure, the recombinant collagen is expressed by a human embryonic kidney cell Expi293F at a high protein expression level, thereby realizing the expression of a large-molecular-weight soluble recombinant collagen.

Activity studies have shown that the expressed recombinant collagen has desirable activity in promoting cell migration, and shows high stability at 4° C.

### BRIEF DESCRIPTION OF THE DRAWINGS

FIG. 1 shows a sodium dodecyl sulfate-polyacrylamide gel electrophoresis (SDS-PAGE) diagram of the recombinant collagen III+I in the present disclosure;

FIG. 2 shows an experimental result of a cell migration promoted by the recombinant collagen III+I in the present disclosure;

FIG. 3 shows a comparative result of activity in promoting cell migration by the recombinant type III collagen derived from different hosts; and

FIG. 4 shows an experimental result of stability of the recombinant collagen III+I in the present disclosure.

### DETAILED DESCRIPTION OF THE EMBODIMENTS

The present disclosure provides a recombinant collagen, including a part of amino acid sequences of a type III human collagen and a part of amino acid sequences of an α2 chain of a type I human collagen.

In the present disclosure, the part of amino acid sequences of the type III human collagen includes preferably amino acid sequences at positions 847 to 849 of the type III human collagen; and the part of amino acid sequences of the α2 chain of the type I human collagen includes amino acid sequences at positions 80 to 1,119 of the α2 chain of the type I human collagen. In the present disclosure, the recombinant collagen further includes preferably a secretion tag, a histidine tag, a fusion tag, and a protease cleavage site sequence.

In the present disclosure, the secretion tag is preferably selected from the group consisting of trypsin (SEQ ID NO: 2), human interleukin 2 (SEQ ID NO: 3), serum albumin (SEQ ID NO: 4), disulfide-bond formation protein A (DsbA, SEQ ID NO: 5), pectate lyase B (PelB, SEQ ID NO: 6), outer membrane protein A (OmpA, SEQ ID NO: 7), maltose-binding protein (SEQ ID NO: 8, whose coding gene is MalE), murein lipoprotein (lpp, SEQ ID NO: 9), trimethylamine N-oxide reductase (TorA, SEQ ID NO: 10), mannosidase (SEQ ID NO: 11), human insulin (SEQ ID NO: 12), and a hybrid secretion tag; the hybrid secretion tag includes a part of a first secretion tag fused to a part of a second secretion tag. In an example, the mannosidase is used as a secretion tag for illustration, but cannot be regarded as the entire protection scope of the present disclosure.

In the present disclosure, the fusion tag is preferably selected from the group consisting of maltose-binding protein (MBP, SEQ ID NO: 13), cysteine protease (CPD, SEQ ID NO: 14), glutathione S-transferase (GST, SEQ ID NO: 15), apolipoprotein A1 (APOI, SEQ ID NO: 16), thioredoxin (TrxA, SEQ ID NO: 17), green fluorescent protein (GFP, SEQ ID NO: 18), and a hybrid fusion tag. The hybrid fusion tag includes a part of a first fusion tag fused to a part of a second fusion tag. In an example, the GFP is used as a fusion tag for illustration, but cannot be regarded as the entire protection scope of the present disclosure.

In the present disclosure, the protease cleavage site sequence is preferably selected from the group consisting of a tobacco etch virus cleavage sequence (TEV, SEQ ID NO: 19), a thrombin cleavage sequence (SEQ ID NO: 20), an enterokinase cleavage sequence (EK, SEQ ID NO: 21), a factor Xa cleavage sequence (Factor-Xa, SEQ ID NO: 22), and a rhinovirus 3C cleavage sequence (HRV3C, SEQ ID NO: 23). In an example, the TEV is used as a protease cleavage site sequence for illustration, but cannot be regarded as the entire protection scope of the present disclosure.

In an example of the present disclosure, the recombinant collagen has an amino acid sequence preferably shown in SEQ ID NO: 1, where the secretion tag and the fusion tag enable a human embryonic kidney cell Expi293F to secrete and express soluble recombinant collagen and increase expression efficiency and protein stability. A protease recognizes the protease cleavage site sequence and removes the fusion tag.

The present disclosure further provides a recombinant expression vector, including an encoding gene of the recombinant collagen.

In the present disclosure, pcDNA3.1 is preferably used as a base vector for the recombinant expression vector, and the encoding gene is inserted between two cleavage sites NheI and EcoRI of the base vector. In an example, the inserted encoding gene has a nucleotide sequence preferably shown in SEQ ID NO: 24. There is no special limitation on the construction method of the recombinant expression vector, which can be constructed by a conventional vector construction method in the art.

The present disclosure further provides an expression method of the recombinant collagen, including: transforming the recombinant expression vector into a human embryonic kidney cell Expi293F to allow culture and expression.

In the present disclosure, there is no special limitation on a transformation method, which can be conducted by conventional transformation methods in the art, such as a plasmid transfection kit. Escherichia coli cells BL21(DE3) express the amino acid sequences at positions 154 to 1,221 (including 1,068 amino acids, a carrier is pET28a(+)) of an α chain of the recombinant human type III collagen. The results show that the recombinant human type III collagen is only expressed in inclusion bodies, with a poor expression level. The inclusion bodies are generally misfolded proteins, which are substantially inactive, have poor water solubility, and are difficult to purify. In addition, the results of a cell migration activity experiment show that: compared with commercially available recombinant type III human collagen expressed by yeast or Escherichia coli, the recombinant type III human collagen expressed by human cells (Expi293F) has better ability to promote cell migration. This indicates that the human cell expression system is conducive to the expression of a highly-active and large-molecular-weight recombinant collagen.

The present disclosure further provides use of the recombinant collagen or a recombinant collagen prepared by the expression method in preparation of a product for promoting a cell migration activity.

In an example of the present disclosure, the recombinant collagen is purified to verify its activity, and it is confirmed that the expressed recombinant collagen has desirable activity in promoting cell migration.

The present disclosure further provides use of the recombinant collagen or a recombinant collagen prepared by the expression method in preparation of a skin repair product.

In order to further illustrate the present disclosure, the recombinant collagen, the expression method and, the use thereof provided in the present disclosure are described in detail below with reference to examples, but the examples should not be interpreted as a limitation to the protection scope of the present disclosure.

### Example 1

### Expression of Recombinant Collagen III+I

Company-synthesized mannosidase secretion tag, GFP fusion tag, TEV protease cleavage site sequence, and recombinant collagen III+I (DNA sequence: SEQ ID NO: 24) were inserted into two cleavage sites NheI and EcoRI of a plasmid pcDNA3.1 to obtain an expression vector capable of expressing the recombinant collagen III+I.

Expi293F cells were subjected to suspension culture in a shaker with a serum-free medium (Genetimes Excell: HE000-N012) at 37° C., 110 rpm, humidity 80%, carbon dioxide 7%. When a cell density reached (2.5-3.0)×10⁶ cells/mL (viable cell rate >95%), transient transfection of the Expi293F cells was conducted using a plasmid transfection kit (Beyotime: C0518) (taking 40 mL as an example).

80 μL of a transfection reagent was added to 1 mL of a cell medium, and mixed gently to obtain solution A; 40 μg of the plasmid was added to 1 mL of the cell medium, and mixed gently to obtain solution B. The solution B was gently mixed with the solution A and allowed to stand for 15 min. A mixture of A and B was added to 40 mL of the Expi293F cells obtained by the suspension culture, and the culture was continued for 6 d to 7 d, and the cells were collected when the viable cell rate was not more than 50%.

A resulting cell suspension was centrifuged at 5,000 rpm for 10 min at 4° C., the obtained supernatant was incubated with an equilibrated 2 mL nickel column (Thermo Fisher: 88221), eluted by gravity, gradient-eluted with PBS solutions containing 10 mM, 30 mM, 50 mM, 100 mM, 200 mM, and 300 mM imidazole separately, and the protein was detected by SDS-PAGE; the obtained PBS eluates containing the target protein with high purity were combined, and imidazole was removed by an ultrafiltration tube (Millipore, UFC9010). 200 μL of PBS solution containing GFP-fused recombinant collagen was added to 20 μL of enzyme reaction solution (Beyotime: P2307), and then 50 U of enzyme solution was added to allow reaction overnight at 4° C., and the reaction was detected by SDS-PAGE. The results are shown in FIG. 1. A protein mixture solution was incubated with the nickel column to remove the tagged protein and enzyme, and the penetration solution contained the recombinant collagen III+I. As shown in FIG. 1, the target protein had a molecular weight of about 150 KDa; the nickel column could enrich the target protein, and the enriched protein showed green fluorescence; the TEV enzyme could cleave the fusion tag of the target protein. These properties proved that the expressed target protein had an amino acid sequence corresponding to the target gene, and the protein expression level was 1.2 mg/40 mL of cells.

### Example 2

### Determination of Activity of the Recombinant Collagen III+I in Promoting Cell Migration

On the back of a 6-well plate, horizontal lines were evenly drawn with a marker using a ruler, where one horizontal line was drawn every 0.5 cm to 1 cm, and 5 horizontal lines were drawn for each well. About 5×10⁵ BALB/c 3T3 cells were added to each well and cultured overnight to make the cells adhere to the wall. The next day, the cells were streaked with a sterile tip using a ruler, and washed 3 times with PBS to remove the streaked cells; a serum-free DMEM medium was added to set up a control group and a recombinant collagen experimental group (100 μg/mL), and the cells were returned to the incubator. Sampling and photo-taking were conducted at 0 h, 6 h, 12 h, and 24 h.

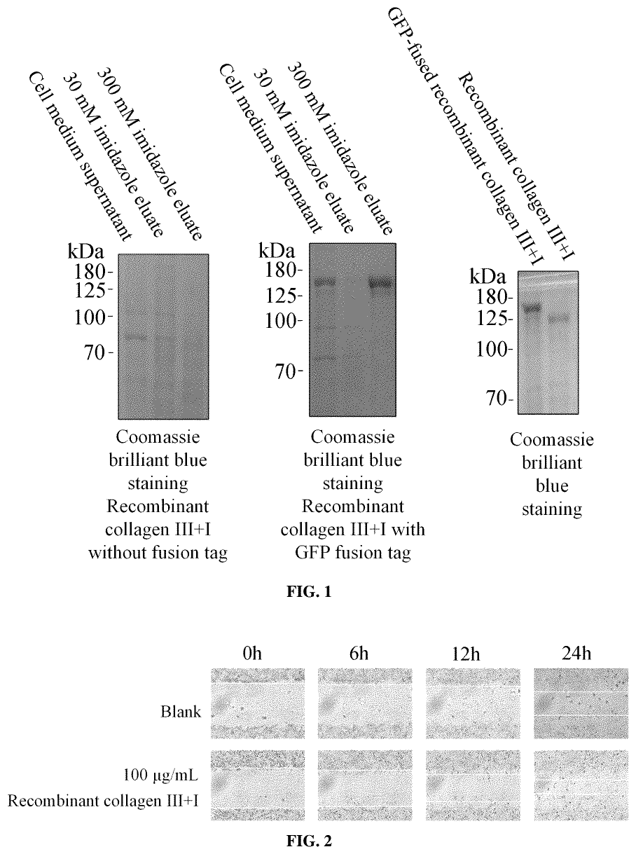

The results are shown in FIG. 2. Compared with the control group, 100 μg/mL recombinant collagen III+I had the ability to significantly promote the migration of BALB/c 3T3 cells.

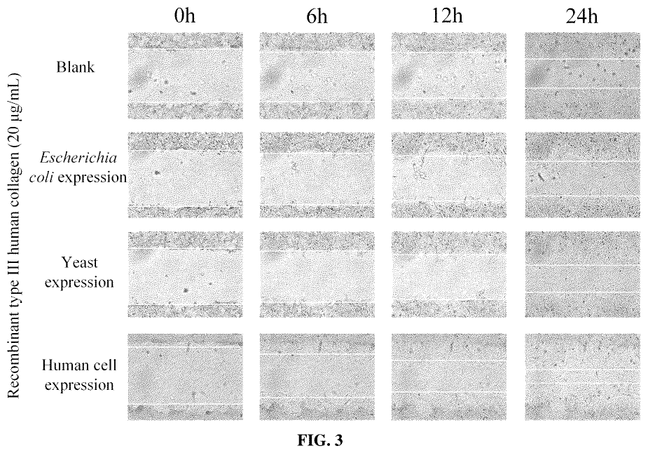

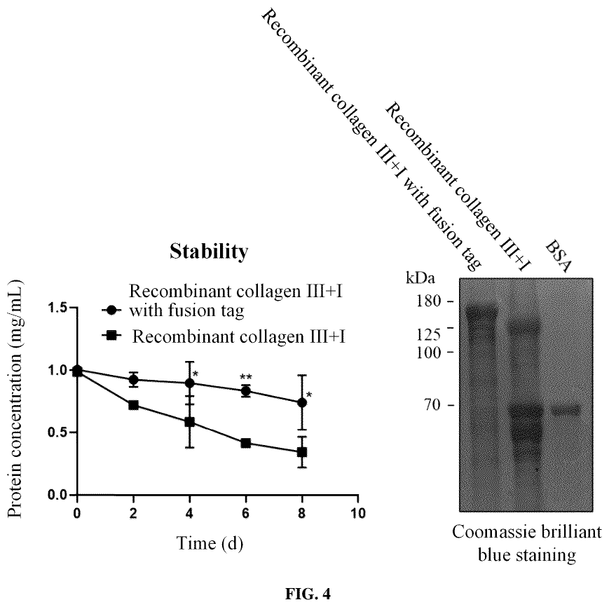

The results are shown in FIG. 3. Compared with the control group, the recombinant III collagen expressed by 20 μg/mL Escherichia coli basically had no activity in promoting cell migration. The recombinant III collagen expressed by yeast and human cells had better activity in promoting cell migration, and the recombinant III collagen expressed by human cells showed the best activity.

### Example 3

Experimental Research on Stability of Recombinant Collagen III+I with Fusion Tag Prepared in Example 1 and Recombinant Collagen III+I

The recombinant collagen III+I with fusion tag and the recombinant collagen III+I were separately prepared into 1 mg/mL PBS solution and stored in a refrigerator at 4° C.; 5 μL (5 μg) of each sample was taken out every 2 d (48 h), into which 5 μL of SDS-PAGE protein loading buffer (2×) (Beyotime: P0015B) was added; the changes in protein amount and position were detected by SDS-PAGE, with BSA set as a control (2 μg).

The results are shown in FIG. 4. Compared with the recombinant collagen III+I, the recombinant collagen III+I with fusion tag had a better stability, where P<0.05 was * (statistically significant difference), and P<0.01 was **. The two groups of proteins on day 8 were analyzed by SDS-PAGE, and the results showed that the recombinant collagen III+I had obvious degradation protein bands.

Although the above examples have described the present disclosure in detail, they are only a part of, not all of, the examples of the present disclosure. Other examples may also be obtained by a person of ordinary skill based on the example without creative efforts, and all of these examples shall fall within the protection scope of the present disclosure.

1-10. (canceled)

11. A recombinant collagen, comprising all or a part of amino acid sequences of a type III human collagen and all or a part of amino acid sequences of a type I human collagen.

12. The recombinant collagen according to claim 11, wherein the part of amino acid sequences of the type III human collagen comprises amino acid sequences at positions 847 to 849 or at positions 154 to 1,221 of the type III human collagen; and
the part of amino acid sequences of the type I human collagen comprises amino acid sequences at positions 80 to 1,119 of an α2 chain of the type I human collagen.

13. The recombinant collagen according to claim 11, further comprising a secretion tag, a histidine tag, a fusion tag, and a protease cleavage site sequence.

14. The recombinant collagen according to claim 13, wherein the secretion tag is selected from the group consisting of trypsin, human interleukin 2, serum albumin, disulfide-bond formation protein A, pectate lyase B, outer membrane protein A, maltose-binding protein, murein lipoprotein, trimethylamine N-oxide reductase, mannosidase, human insulin, and a hybrid secretion tag:
the fusion tag is selected from the group consisting of maltose-binding protein (MBP), cysteine protease, glutathione S-transferase, apolipoprotein A1, thioredoxin, green fluorescent protein, and a hybrid fusion tag; and
the protease cleavage site sequence is selected from the group consisting of a tobacco etch virus cleavage sequence, a thrombin cleavage sequence, an enterokinase cleavage sequence, a factor Xa cleavage sequence, a rhinovirus 3C cleavage sequence, and a hybridase cleavage site sequence.

15. The recombinant collagen according to claim 13, wherein the recombinant collagen comprises an amino acid sequence shown in SEQ ID NO: 1.

16. The recombinant collagen according to claim 14, wherein the recombinant collagen comprises an amino acid sequence shown in SEQ ID NO: 1.

17. A recombinant expression vector, comprising an encoding gene of the recombinant collagen according to claim 11.

18. The recombinant expression vector according to claim 17, wherein the part of amino acid sequences of the type III human collagen comprises amino acid sequences at positions 847 to 849 or at positions 154 to 1,221 of the type III human collagen; and
the part of amino acid sequences of the type I human collagen comprises amino acid sequences at positions 80 to 1,119 of an α2 chain of the type I human collagen.

19. The recombinant expression vector according to claim 17, further comprising a secretion tag, a histidine tag, a fusion tag, and a protease cleavage site sequence.

20. The recombinant expression vector according to claim 19, wherein the secretion tag is selected from the group consisting of trypsin, human interleukin 2, serum albumin, disulfide-bond formation protein A, pectate lyase B, outer membrane protein A, maltose-binding protein (MalE), murein lipoprotein, trimethylamine N-oxide reductase, mannosidase, human insulin, and a hybrid secretion tag:
the fusion tag is selected from the group consisting of maltose-binding protein (MBP), cysteine protease, glutathione S-transferase, apolipoprotein A1, thioredoxin, green fluorescent protein, and a hybrid fusion tag; and
the protease cleavage site sequence is selected from the group consisting of a tobacco etch virus cleavage sequence, a thrombin cleavage sequence, an enterokinase cleavage sequence, a factor Xa cleavage sequence, a rhinovirus 3C cleavage sequence, and a hybridase cleavage site sequence.

21. The recombinant expression vector according to claim 17, wherein the recombinant collagen comprises an amino acid sequence shown in SEQ ID NO: 1.

22. The recombinant expression vector according to claim 17, wherein the encoding gene comprises a nucleotide sequence shown in SEQ ID NO: 24.

23. A method for expressing the recombinant collagen according to claim 11, comprising:
transforming a recombinant expression vector comprising an encoding gene of the recombinant collagen into a human embryonic kidney cell Expi293F; and
culturing and expressing the human embryonic kidney cell Expi293F.

24. A method for promoting migration activity of a cell, comprising applying the recombinant collagen according to claim 11 to the cell.

25. The method according to claim 24, wherein the part of amino acid sequences of the type III human collagen comprises amino acid sequences at positions 847 to 849 or at positions 154 to 1,221 of the type III human collagen; and
the part of amino acid sequences of the type I human collagen comprises amino acid sequences at positions 80 to 1,119 of an α2 chain of the type I human collagen.

26. The method according to claim 24, further comprising a secretion tag, a histidine tag, a fusion tag, and a protease cleavage site sequence.

27. The method according to claim 26, wherein the secretion tag is selected from the group consisting of trypsin, human interleukin 2, serum albumin, disulfide-bond formation protein A, pectate lyase B, outer membrane protein A, maltose-binding protein, murein lipoprotein, trimethylamine N-oxide reductase, mannosidase, human insulin, and a hybrid secretion tag:
the fusion tag is selected from the group consisting of maltose-binding protein (MBP), cysteine protease, glutathione S-transferase, apolipoprotein A1, thioredoxin, green fluorescent protein, and a hybrid fusion tag; and
the protease cleavage site sequence is selected from the group consisting of a tobacco etch virus cleavage sequence, a thrombin cleavage sequence, an enterokinase cleavage sequence, a factor Xa cleavage sequence, a rhinovirus 3C cleavage sequence, and a hybridase cleavage site sequence.

28. The method according to claim 24, wherein the recombinant collagen comprises an amino acid sequence shown in SEQ ID NO: 1.

29. A method for repairing skin, comprising applying the recombinant collagen according to claim 11 to the skin.

30. The method according to claim 29, wherein the part of amino acid sequences of the type III human collagen comprises amino acid sequences at positions 847 to 849 or at positions 154 to 1,221 of the type III human collagen; and
the part of amino acid sequences of the type I human collagen comprises amino acid sequences at positions 80 to 1,119 of an α2 chain of the type I human collagen.
